(19) United States
(12) Patent Application Publication
(10) Pub. No.: US 2022/0079886 A1
(43) Pub. Date: Mar. 17, 2022

(54) METHOD OF PREPARATION OF BIOCOMPATIBLE METAL NANOPARTICLES AND APPLICATIONS THEREOF

(71) Applicant: Oceanit Laboratories Inc., Honolulu, HI (US)

(72) Inventors: Kathryn ANDERSON, Waianae, HI (US); Ganesh ARUMUGAM, Honolulu, HI (US); Dexter POON, Honolulu, HI (US)

(21) Appl. No.: 17/024,596

(22) Filed: Sep. 17, 2020

Related U.S. Application Data
(60) Provisional application No. 62/901,751, filed on Sep. 17, 2019.

(51) Int. Cl. A61K 9/51; A61K 33/242; A61K 47/18

(52) U.S. Cl. CPC A61K 9/5192; A61K 33/242; B82Y 5/00; A61K 9/5146; A61K 9/5123; A61K 47/183

(57) ABSTRACT

The present invention is generally directed to biocompatible metal nanoparticles comprising a plurality of metal nanoparticles covalently bonded to a plurality of biocompatible functional ligands. The plurality of metal nanoparticles may be chosen from any suitable metal nanoparticle, including, but not limited to, gold. The plurality of biocompatible functional ligands may comprise at least one amino acid selected from the group consisting of proline, hydroxyproline, glycine, cysteine, and combinations thereof. Embodiments of the present invention may also be directed towards methods of synthesizing biocompatible metal nanoparticles. Metal nanoparticles, such as gold nanoparticles, are covalently bonded to citrate, resulting in gold-citrate nanoparticles, each of which will be tailored to 5-200 nm. Various amino acids may then be functionalized on to the gold-citrate nanoparticles, including, without limitation, three amino acids present in collagen (i.e., proline, hydroxyproline, and glycine), as well as cysteine. The amino acids may be functionalized through a variety of methods, including the use of thiol functional ligands and carboxylic acid functional ligands.

### FIELD OF THE INVENTION

The application relates generally to methods, products, and systems for preparing biocompatible metal nanoparticles, which are usable in a variety of industries, including, but not limited to, the beauty and makeup industry, skincare applications, food and medical applications, the healthcare industry, and the like.

### BACKGROUND

Usage of amino acids in a variety of applications, including, but not limited to, beauty and makeup, skincare, and other medical applications may be beneficial. Various proteins and peptides may promote health, but are less likely to penetrate skin layers due to steric hinderance. For instance, collagen is unlikely to penetrate the stratum corneum outer skin layer because it has, on average, a size of 45,000 Daltons, which is substantially larger than the empirical rule of 500 Da as the maximum size for dermal penetration.

Metal nanoparticles, such as, for example, gold nanoparticles, have been shown to be feasible as a delivery vehicle for drugs and therapeutic agents in the human body. A dermal penetration study using gold nanoparticles, published by Schneider et al., reported aggregation in deep skin layers of rats when these nanoparticles were below 100 nm in diameter. (See Schneider et al., Nanoparticles and their interactions with the dermal barrier, Dermato-Endocrinology 1:4, 197-206 (July/August 2009), available at https://www.ncbi.nlm.nih.gov/pmc/articles/PMC2835875/pdf/de0104_0197.pdf, which is hereby incorporated by reference in its entirety).

Collagen is used in skin care products to stimulate the fibroblasts in the skin to produce more collagen and elastin, thereby bringing natural hydration to the area and firming the skin, thus reducing wrinkles and improving skin health. Collagen is a protein comprised of three left handed polypeptide chains to make a right handed triple helix. The fibrillar structure of type I collagen, which is the prototypical collagen fibril, is a sequence of three amino acids, XaaYaaGly, where Xaa and Yaa can be (2S)-proline and (2S,4R)-4-hydroxyproline, respectively. (See Shoulders et al., Collagen Structure and Stability, Ann. Rev. Biochem. 2009; 78:929-958, available at https://www.annualreviews.org/doi/10.1146/annurev.biochem.77.032207.120833, which is hereby incorporated by reference in its entirety).

An amino acid that stabilizes collagen and is beneficial for skin and organ health is L-cysteine. L-cysteine, found in beta-keratin (the human protein in nails, hair and skin), is a non-essential and sulfur containing amino acid. L-cysteine has anti-aging properties due to its role in the process of detoxification and the synthesis of glutathione in the body, both of which result in the protection of several tissues and organs.

Given the foregoing, there exists a significant need for a new technology capable of allowing for biocompatible metal nanoparticles using a metal carrier, such as gold, attached to beneficial amino acids, such as collagen amino acids.

### SUMMARY

It is to be understood that both the following summary and the detailed description are exemplary and explanatory and are intended to provide further explanation of the invention as claimed. Neither the summary nor the description that follows is intended to define or limit the scope of the invention to the particular features mentioned in the summary or in the description.

In general, the present invention is directed to biocompatible metal nanoparticles comprising a plurality of metal nanoparticles covalently bonded to a plurality of biocompatible functional ligands. The plurality of metal nanoparticles may be chosen from any suitable metal nanoparticle, including, but not limited to, gold. The plurality of biocompatible functional ligands may comprise at least one amino acid selected from the group consisting of proline, hydroxyproline, glycine, cysteine, and combinations thereof. Covalent attachment of beneficial amino acids, including, without limitation, amino acids present in collagen (or “collagen amino acids”) to gold nanoparticles allows delivery of such amino acids deep within skin to promote skin health.

Gold nanoparticles are commonly produced in industry by the following methods: (1) citrate reduction of HAuCl₄, (2) the Brust-Schiffrin method using tetraoctylamonium bromide with sodium borohydrate in toluene with HAuCl₄, (3) the Perrault method using hydroquinone and HAuCl₄, (4) the Martin method using sodium borohydrate, HAuCl₄, and a ratio of HCl/NaOH, or (5) the Turkevich method that uses fewer toxic materials. These methods, or other biocompatible methods using ascorbic acid, may be employed to create the gold nanoparticles, where the diameter size of each resulting gold-citrate nanoparticle is tailored to less than 100 nm. (See Tyagi et al., “pH-dependent synthesis of stabilized gold nanoparticles using ascorbic acid,” Int. J. Neuroscience 10:857 (August 2011), available at https://www.researchgate.net/publication/235999581_PH-dependent_synthesis_of_stabilized_gold_nanoparticles_using_ascorbic_acid, which is herein incorporated by reference in its entirety).

Embodiments of the present invention include grafting various amino acids to the metal nanoparticles. In one embodiment of the invention, various amino acids present in collagen (“collagen amino acids”) are used. Collagen is used in skin care products to stimulate the fibroblasts in the skin to produce more collagen to create healthier, firmer skin, thus reducing wrinkles. Collagen is a protein comprised of three left handed polypeptide chains to make a right handed triple helix. The fibrils can be broken down into three basic amino acid repeat units of 2S-proline (or “proline”), glycine, and (2S,4R)-4-hydroxyproline (or “hydroxyproline”).

Another amino acid that stabilizes collagen and is beneficial for skin and organ health is L-cysteine. L-cysteine, found in beta-keratin (the human protein found in nails, hair and skin), is a non-essential and sulfur-containing amino acid. L-cysteine (or “cysteine”) has anti-aging properties due to its role in the process of detoxification and the synthesis of glutathione in the body, both of which result in the protection of several tissues and organs. According to recent research, cysteine not only slows down the natural process of ageing, but also helps in preventing certain diseases. These include dementia and multiple sclerosis, since research suggests a link between these diseases and an accumulation of toxins.

These and further and other objects and features of the invention are apparent in the disclosure, which includes the above and ongoing written specification, as well as the drawings.

### BRIEF DESCRIPTION OF THE DRAWINGS

The accompanying drawings, which are incorporated herein and form a part of the specification, illustrate exemplary embodiments and, together with the description, further serve to enable a person skilled in the pertinent art to make and use these embodiments and others that will be apparent to those skilled in the art.

FIG. 1 illustrates the structure of a collagen fibril and the sequence of relevant amino acids, according to an embodiment of the present invention.

FIG. 2 illustrates the development of functional ligands using collagen amino acids.

FIGS. 3A-3C illustrate various functionalization reactions for gold-citrate nanoparticles.

FIGS. 4A-4B illustrate various secondary coupling reactions with carboxylic acid functionalized amino acid ligands.

### DETAILED DESCRIPTION

The present invention is more fully described below with reference to the accompanying figures. The following description is exemplary in that several embodiments are described (e.g., by use of the terms “preferably,” “for example,” or “in one embodiment”); however, such should not be viewed as limiting or as setting forth the only embodiments of the present invention, as the invention encompasses other embodiments not specifically recited in this description, including alternatives, modifications, and equivalents within the spirit and scope of the invention. Further, the use of the terms “invention,” “present invention,” “embodiment,” and similar terms throughout the description are used broadly and not intended to mean that the invention requires, or is limited to, any particular aspect being described or that such description is the only manner in which the invention may be made or used. Additionally, the invention may be described in the context of specific applications; however, the invention may be used in a variety of applications not specifically described.

In the several figures, like reference numerals may be used for like elements having like functions even in different drawings. The embodiments described, and their detailed construction and elements, are merely provided to assist in a comprehensive understanding of the invention. Thus, it is apparent that the present invention can be carried out in a variety of ways, and does not require any of the specific features described herein. Also, well-known functions or constructions are not described in detail since they would obscure the invention with unnecessary detail. Any signal arrows in the drawings/figures should be considered only as exemplary, and not limiting, unless otherwise specifically noted. Further, the description is not to be taken in a limiting sense, but is made merely for the purpose of illustrating the general principles of the invention, since the scope of the invention is best defined by the appended claims.

It will be understood that, although the terms first, second, etc. may be used herein to describe various elements, these elements should not be limited by these terms. These terms are only used to distinguish one element from another. Purely as a non-limiting example, a first element could be termed a second element, and, similarly, a second element could be termed a first element, without departing from the scope of example embodiments. As used herein, the term “and/or” includes any and all combinations of one or more of the associated listed items. As used herein, the singular forms “a”, “an,” and “the” are intended to include the plural forms as well, unless the context clearly indicates otherwise. It should also be noted that, in some alternative implementations, the functions and/or acts noted may occur out of the order as represented in at least one of the several figures. Purely as a non-limiting example, two figures shown in succession may in fact be executed substantially concurrently or may sometimes be executed in the reverse order, depending upon the functionality and/or acts described or depicted.

Generally, the present invention is directed to biocompatible metal nanoparticles comprising a plurality of metal nanoparticles covalently bonded to a plurality of biocompatible functional ligands. The plurality of metal nanoparticles may be chosen from any suitable metal nanoparticle, including, but not limited to, gold. The plurality of biocompatible functional ligands may comprise at least one amino acid selected from the group consisting of proline, hydroxyproline, glycine, cysteine, and combinations thereof.

Gold nanoparticles, are commonly produced in industry by the following methods: (1) citrate reduction of HAuCl₄, (2) the Brust-Schiffrin method using tetraoctylamonium bromide with sodium borohydrate in toluene with HAuCl₄, (3) the Perrault method using hydroquinone and HAuCl₄, (4) the Martin method using sodium borohydrate, HAuCl₄, and a ratio of HCl/NaOH, or (5) the Turkevich method that uses fewer toxic materials. These methods may be employed to create the gold nanoparticles, whose diameter size of each resulting gold-citrate nanoparticle is tailored to less than 100 nm, or by other biocompatible methods using ascorbic acid.

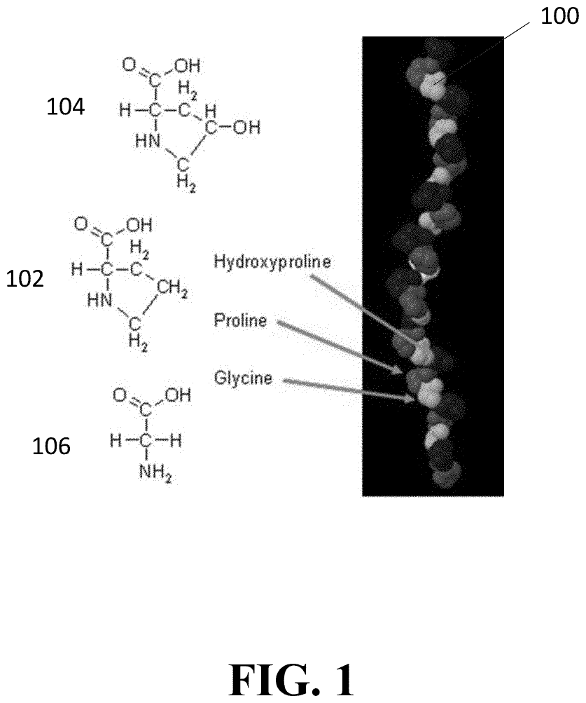

Various amino acids may then be functionalized on to the gold-citrate nanoparticles, including, without limitation, three amino acids present in collagen (i.e., proline, hydroxyproline, and glycine), as well as cysteine. The structure of a collagen fibril 100 is shown in FIG. 1. Also shown are the structures of proline 102, hydroxyproline 104, and glycine 106. These amino acids, as well as cysteine, may be functionalized through a variety of methods.

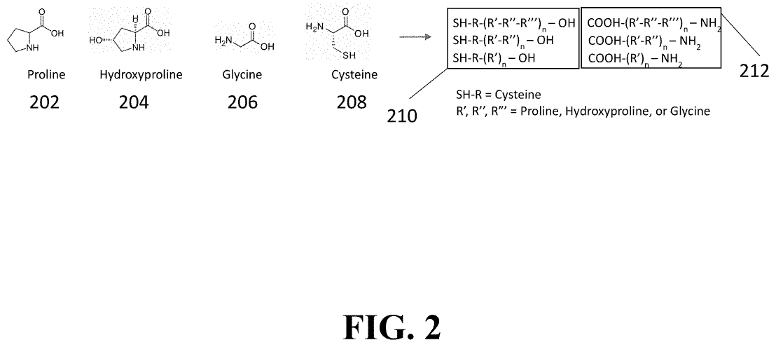

One such method is through the use of thiolated ligands, which have been extensively used to functionalize the surface of gold. In one embodiment of the invention, illustrated in FIG. 2, one or more of the collagen amino acids (i.e., proline 202, hydroxyproline 204, and glycine 206) are reacted with cysteine 208 to create thiol functional ligands 210, which can directly react with metal nanoparticles, including, for example, gold nanoparticles.

In another embodiment of the invention, also illustrated in FIG. 2, the collagen amino acids (proline 202, hydroxyproline 204, and glycine 206) and/or cysteine 208 are reacted in sequence, using coupling agents and deprotection steps commonly known in the art, to expose functional carboxylic acid groups 212, which can react with an anime-capped metal nanoparticle, such as, for example, an anime-capped gold nanoparticle.

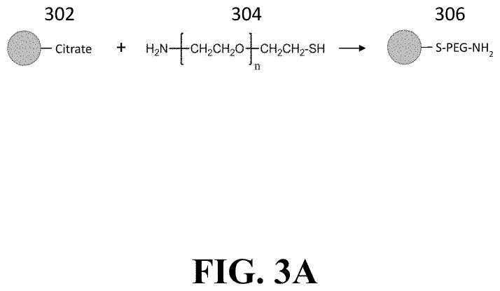

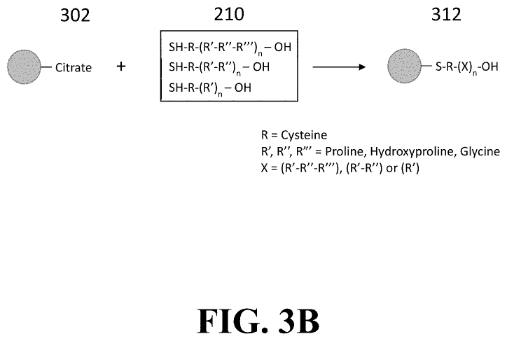

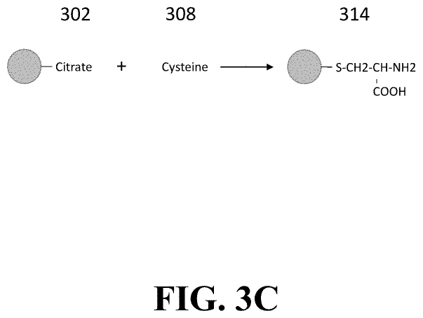

FIGS. 3A-3C illustrate further details of the functionalization reactions of gold-citrate nanoparticles. In FIG. 3A, gold-citrate nanoparticles 302 are functionalized with thiol-polyethylene glycol (PEG)-amine groups 304 to create PEGylated gold nanoparticles 306 with amine functional groups. In FIG. 3B, gold-citrate nanoparticles 302 are functionalized with the thiol functional ligands 210 as described above to create amino-acid functionalized gold nanoparticles 312. In FIG. 3C, gold-citrate nanoparticles 302 are functionalized with cysteine 308 to create gold-cysteine nanoparticles 314 (also known in the art as “gold-capped cysteine nanoparticles”).

Ideally, a one-step functionalization, such as shown in FIG. 3B, would be ideal to obtain the final biocompatible metal nanoparticle, such as, for example, the amino-acid functionalized gold nanoparticle 312 shown in FIG. 3B. Otherwise, a secondary coupling step may be required.

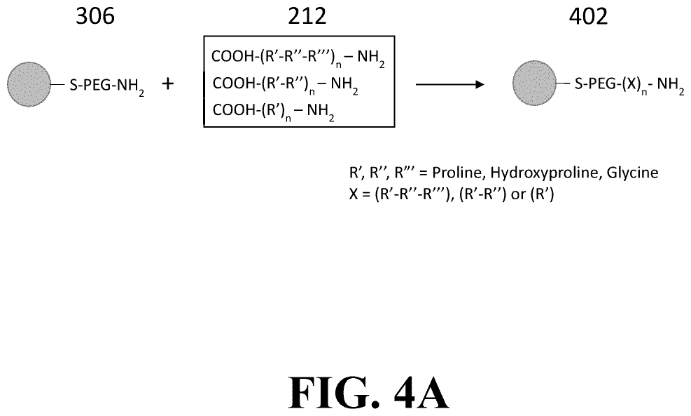

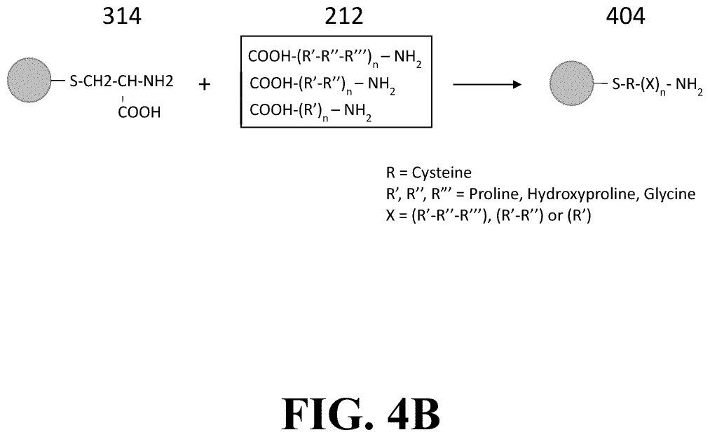

Such secondary reactions are shown in FIGS. 4A and 4B. In FIG. 4A, a secondary reaction is depicted involving the PEGylated gold nanoparticles 306, created according to the reaction illustrated in FIG. 3A. Such PEGylated gold nanoparticles are reacted with carboxylic acid functional ligands 212, created according to the reactions illustrated in FIG. 2. The resultant is a biocompatible metal nanoparticle 402 containing a PEG group as well as one or more amino acids selected from the group consisting of proline, hydroxyproline, glycine, cysteine, and combinations thereof. In FIG. 4B, an alternative secondary reaction involving the gold-cysteine nanoparticles 314, created according to the reaction illustrated in FIG. 3C, is shown. Such gold-cysteine nanoparticles are reacted with carboxylic acid functional ligands 212, created according to the reactions illustrated in FIG. 2. The resultant is a biocompatible metal nanoparticle 404 containing one or more amino acids selected from the group consisting of proline, hydroxyproline, glycine, cysteine, and combinations thereof.

The amino acids that are bonded to the metal nanoparticles impart hydrophilicity, thus enabling suspension of the biocompatible metal nanoparticles in an aqueous solution, including, for example, various types of water-based solutions that are compatible for use in lotions, creams, and other methods of application to the skin. Such aqueous solution may aid in delivering the biocompatible metal nanoparticles to various tissues, including, but not limited to, skin. The biocompatible metal nanoparticles have the ability to penetrate the top five layers of the skin, based on previous research in animal models. As a result, the amino acids in the biocompatible metal nanoparticles promote adhesion with collagen typically found in skin care remedies. In addition, these amino acids promote growth of natural collagen in deep skin layers when applied to target areas on the skin. Further, the biocompatible metal nanoparticles deliver collagen deeper into the skin layers through adhesion of collagen in skin care products, improving cell viability and growth of collagen in the deeper skin layers.

The present invention has been described with gold nanoparticles and their attendant functionalization, but it should be appreciated that the invention in its embodiments is applicable to other types of metal nanoparticles, including, but not limited to, silver, copper, platinum, palladium, nickel, and other transition metals.

These and other objectives and features of the invention are apparent in the disclosure, which includes the above and ongoing written specification.

The invention is not limited to the particular embodiments illustrated in the drawings and described above in detail. Those skilled in the art will recognize that other arrangements could be devised. The invention encompasses every possible combination of the various features of each embodiment disclosed. One or more of the elements described herein with respect to various embodiments can be implemented in a more separated or integrated manner than explicitly described, or even removed or rendered as inoperable in certain cases, as is useful in accordance with a particular application. While the invention has been described with reference to specific illustrative embodiments, modifications and variations of the invention may be constructed without departing from the spirit and scope of the invention as set forth in the following claims.

I claim:

1. A method for producing a plurality of biocompatible metal nanoparticles, the method comprising:
obtaining a plurality of gold nanoparticles;
attaching the plurality of gold nanoparticles to a plurality of citrate molecules, thereby producing a plurality of gold-citrate nanoparticles; and
reacting the plurality of gold-citrate nanoparticles with a plurality of biocompatible functional ligands, thereby producing a plurality of biocompatible metal nanoparticles.

2. The method of claim 1, wherein the plurality of biocompatible functional ligands comprises a plurality of thiol ligands comprising a plurality of amino acids selected from the group consisting of proline, hydroxyproline, glycine, cysteine, and combinations thereof.

3. The method of claim 1, wherein the reacting step further comprises:
bonding the plurality of gold-citrate nanoparticles with a plurality of thiol-polyethylene glycol-amine compounds, thereby producing a plurality of pegylated gold nanoparticles.

4. The method of claim 3, further comprising:
functionalizing the plurality of pegylated gold nanoparticles with the plurality of biocompatible functional ligands.

5. The method of claim 4, wherein the plurality of biocompatible functional ligands comprises a plurality of carboxylic acid ligands comprising a plurality of amino acids selected from the group consisting of proline, hydroxyproline, glycine, cysteine, and combinations thereof.

6. The method of claim 1, wherein the reacting step further comprises:
bonding the plurality of gold-citrate nanoparticles with a plurality of cysteine moieties, thereby producing a plurality of gold-cysteine nanoparticles.

7. The method of claim 6, further comprising:
functionalizing the plurality of gold-cysteine nanoparticles with the plurality of biocompatible functional ligands.

8. The method of claim 7, wherein the plurality of biocompatible functional ligands comprises a plurality of carboxylic acid ligands comprising a plurality of amino acids selected from the group consisting of proline, hydroxyproline, glycine, cysteine, and combinations thereof.

9. A biocompatible metal nanoparticle comprising:
a gold nanoparticle; and
at least one amino acid selected from the group consisting of proline, hydroxyproline, glycine, cysteine, and combinations thereof,
wherein the gold nanoparticle and the at least one amino acid are covalently bonded via a sulfur atom, thereby forming a biocompatible metal nanoparticle.

10. The biocompatible metal nanoparticle of claim 2, further comprising at least one polyethylene glycol compound.

11. A method for producing a plurality of biocompatible metal nanoparticles, the method comprising:
attaching a plurality of gold nanoparticles to a plurality of citrate molecules, thereby producing a plurality of gold-citrate nanoparticles; and
functionalizing the gold-citrate nanoparticles with one or more amino acids, thereby producing a plurality of biocompatible metal nanoparticles.

12. The method of claim 11, wherein the one or more amino acids is selected from the group consisting of: proline, hydroxyproline, glycine, cysteine, and combinations thereof.

13. The method of claim 11, wherein the functionalizing step further comprises:
reacting the one or more amino acids with one or more thiolated ligands, thereby producing one or more thiol functional ligands.

14. The method of claim 13, wherein the functionalizing step further comprises:
reacting the one or more thiol functional ligands with the plurality of gold-citrate nanoparticles.

15. The method of claim 11, wherein the functionalizing step further comprises:
reacting the plurality of gold-citrate nanoparticles with one or more thiol-polyethylene glycol (PEG)-amine groups, thereby producing a plurality of PEG-ylated gold nanoparticles.

16. The method of claim 15, wherein the functionalizing step further comprises:
reacting the plurality of PEG-ylated gold nanoparticles with one or more carboxylic acid functional ligands, thereby producing the plurality of biocompatible metal nanoparticles.

17. The method of claim 11, wherein the functionalizing step further comprises:
reacting the plurality of gold-citrate nanoparticles with cysteine, thereby producing gold-capped cysteine nanoparticles.

18. The method of claim 17, wherein the functionalizing step further comprises:
reacting the gold-capped cysteine nanoparticles with one or more carboxylic acid functional ligands, thereby producing the plurality of biocompatible metal nanoparticles.

19. The method of claim 11, further comprising:
introducing the plurality of biocompatible metal nanoparticles into an aqueous solution in order to deliver the plurality of biocompatible metal nanoparticles to one or more human tissues.

20. The method of claim 11, wherein the one or more amino acids is derived from collagen.
